(19) United States
(12) Patent Application Publication
(10) Pub. No.: US 2025/0116820 A1
(43) Pub. Date: Apr. 10, 2025

(54) HERMETICALLY SEALED FIBER OPTICS WITH PROTECTIVE WINDOW

(71) Applicant: Alcon Inc., Fribourg (CH)

(72) Inventors: Alireza Mirsepassi, Irvine, CA (US); Adela Apostol, Laguna Hills, CA (US)

(21) Appl. No.: 18/887,149

(22) Filed: Sep. 17, 2024

Related U.S. Application Data
(60) Provisional application No. 63/587,799, filed on Oct. 4, 2023.

(51) Int. Cl. G02B 6/36; A61B 18/22

(52) U.S. Cl. CPC G02B 6/3624; A61B 18/22; A61B 2018/2222

(57) ABSTRACT

Embodiments disclosed herein provide an optical fiber system. The optical fiber system includes an optical fiber, a distal window, and a proximal assembly. The distal window is disposed on a distal end of the optical fiber. The proximal assembly includes a proximal ferrule and a proximal window. The proximal window and the distal window comprise a sapphire material or a diamond material.

### BACKGROUND

In a wide variety of medical procedures, laser light is used to perform surgery and/or to treat patient anatomy. For example, in laser photoemulsification, a laser probe propagates a laser beam to emulsify and ablate the crystalline lens for cataract removal. In another example, a laser probe may be used to cut the vitreous in a vitrectomy procedure. A laser beam is typically transmitted from a surgical laser system through an optical fiber that proximally terminates in a port adaptor connected to the surgical laser system, and distally terminates in the laser probe, which is manipulated by a surgeon.

In order to effectively transmit laser light, a laser probe requires an optical fiber having good transmission and low attenuation at the specific wavelengths of light that are required for a given procedure. However, there are typically limited choices of materials that effectively transmit certain wavelengths of light. Consequently, an appropriate fiber material must be carefully selected for the specific wavelength(s) of light that are being used during a procedure. Many such materials that have good transmission characteristics, however, are also susceptible to damage from heat and/or moisture that is generated before, during, and/or after a medical procedure. As a result, laser probes implementing such materials may become degraded over time, shortening the useful lifetime of such components.

### SUMMARY

The present disclosure relates generally to optical fibers, and more specifically, to components for energy delivery in surgical laser systems.

Certain embodiments of the present disclosure provide an optical fiber system. The optical fiber system includes an optical fiber, a distal window, and a proximal assembly. The distal window is disposed on a distal end of the optical fiber. The proximal assembly includes a proximal ferrule and a proximal window. The proximal window and the distal window comprise a sapphire material or a diamond material.

Certain embodiments of the present disclosure provide a system. The system includes a laser system and an optical fiber system. The optical fiber system includes an optical fiber, a distal window, and a proximal assembly. The distal window is disposed on a distal end of the optical fiber. The proximal assembly includes a proximal ferrule and a proximal window. The proximal window and the distal window comprise a sapphire material or a diamond material.

The following description and the related drawings set forth in detail certain illustrative features of one or more embodiments, including those described above.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates a side view of a system for generating laser beams for delivery to a surgical target, in accordance with certain embodiments of the present disclosure.

FIG. 2A illustrates an optical fiber system having a connector window within a proximal ferrule, in accordance with certain embodiments of the present disclosure.

FIG. 2B illustrates an optical fiber system having a connector window at a proximal end of a proximal assembly, in accordance with certain embodiments of the present disclosure.

FIG. 3 illustrates a distal end of an optical fiber system without a distal ferrule, in accordance with certain embodiments of the present disclosure.

FIG. 4 illustrates a distal end of an optical fiber system having a distal cap, in accordance with certain embodiments of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Note that, as described herein, a distal end, segment, or portion of a component refers to the end, segment, or portion that is closer to a patient's body during use thereof. On the other hand, a proximal end, segment, or portion of the component refers to the end, segment, or portion that is distanced further away from the patient's body and is in proximity to, for example, a surgical laser system.

As used herein, the term “about” may refer to a +/−10% variation from the nominal value. It is to be understood that such a variation can be included in any value provided herein.

Particular embodiments disclosed herein generally relate to optical fibers, and more specifically, to components for energy delivery in surgical laser systems. The methods and systems described herein may be utilized in combination with any suitable laser surgical systems, such as those described below.

During surgery and/or treating patient anatomy, optical fibers of various types may have transmission properties that allow the optical fibers to efficiently and accurately guide a laser beam from a laser source to the surgical site. However, due to the high power of the laser beam, the optical fibers may experience large thermo-mechanical pressures during operation. For example, the laser beams are emitted from the end of the laser probe, the light may be absorbed by water and other liquids within the patient's eye. The absorption of the light may create a high pressure and temperature environment near the end of the optical fiber. Such micro-explosions, which occur when the high pressure area expands, can damage the end of the optical fiber. Additionally, after a particular procedure is completed, the high heat used during autoclaving of the fiber optic cable can further damage the optical fiber.

By implementing optical fiber materials having a high hardness, the thermo-mechanical damage may be reduced. However, materials having sufficient hardness and resistance to heat and/or moisture may not have sufficient transmission characteristics at the relevant wavelength(s) of light. For instance, surface contamination on a high hardness material can cause the laser light travelling through the optical fiber to be absorbed, leading to inefficiencies.

In various embodiments, the surgical system disclosed herein may be implemented to provide heat and moisture resistance while retaining superior light transmission properties at wavelengths of light used in surgical systems.

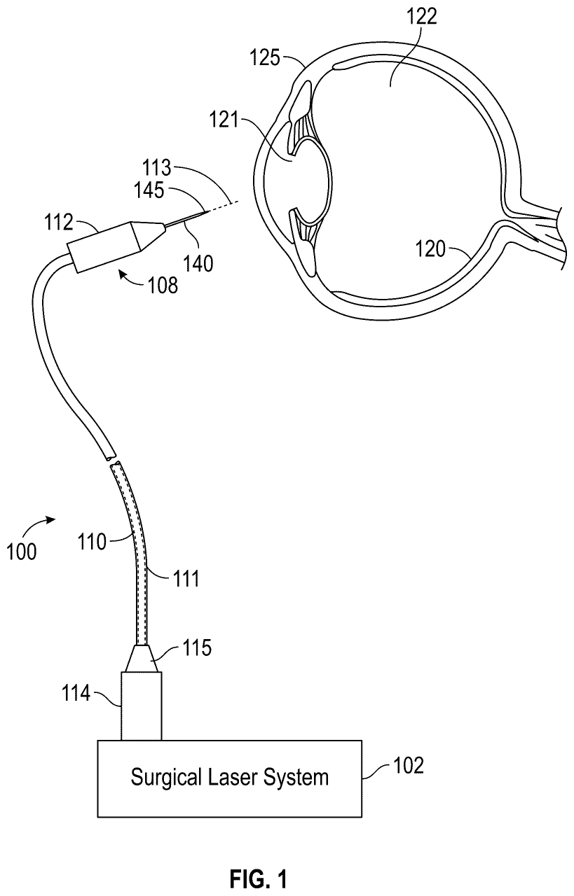

FIG. 1 illustrates an example surgical system 100 for performing a laser-assisted ophthalmic procedure. Surgical system 100 includes a laser system 102, having one or more laser sources for generating a laser beam 113. In one example, the laser beam 113 may have a wavelength of about 1 μm (micrometers) to about 10 μm, such as about 3 μm and may be suitable for, for example, photoemulsification, laser vitrectomy, or other types of tissue removal. In another example, laser beam 113 may have a wavelength appropriate for photocoagulation to treat retinal tears and detachments, etc. The user, such as a surgeon, may toggle the laser between on positions and off positions using a switch on a probe 108, a foot pedal, or other means. The probe 108 is meant to be representative of a probe that may be used in a variety of surgical systems, such as for photocoagulation in retinal surgery, photoemulsification in cataract surgery, vitrectomy, or other surgical procedures. For example, the surgeon may activate a laser beam, such as through a foot pedal or other means, to emulsify and remove the patient's cataract, cut and remove the patient's vitreous, perform photocoagulation, or the like. In further examples, the laser system 102 includes an illumination system including one or more illumination sources.

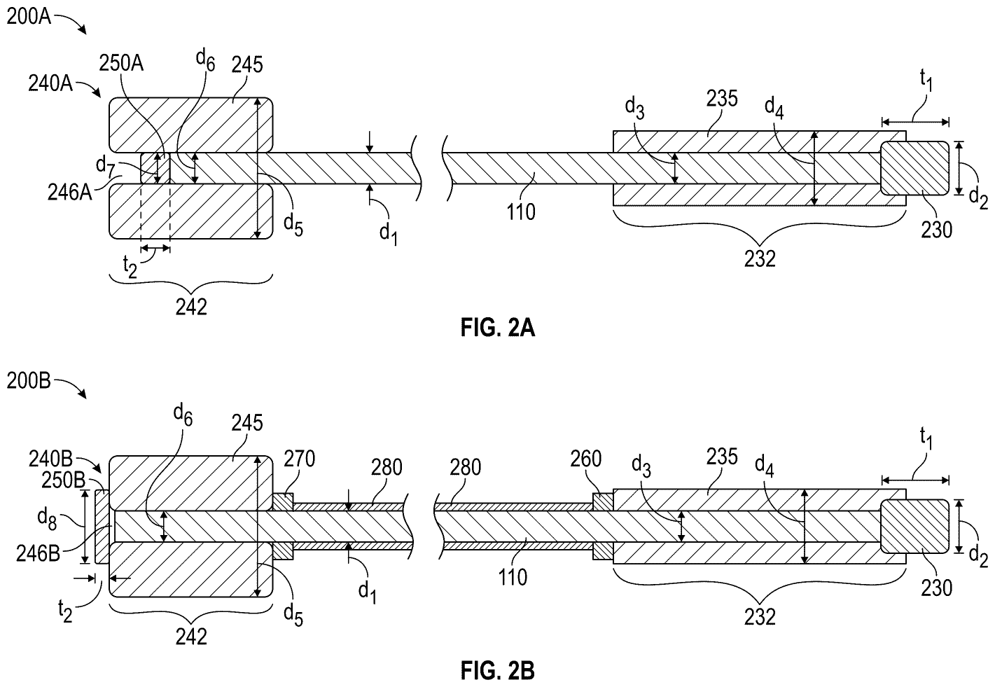

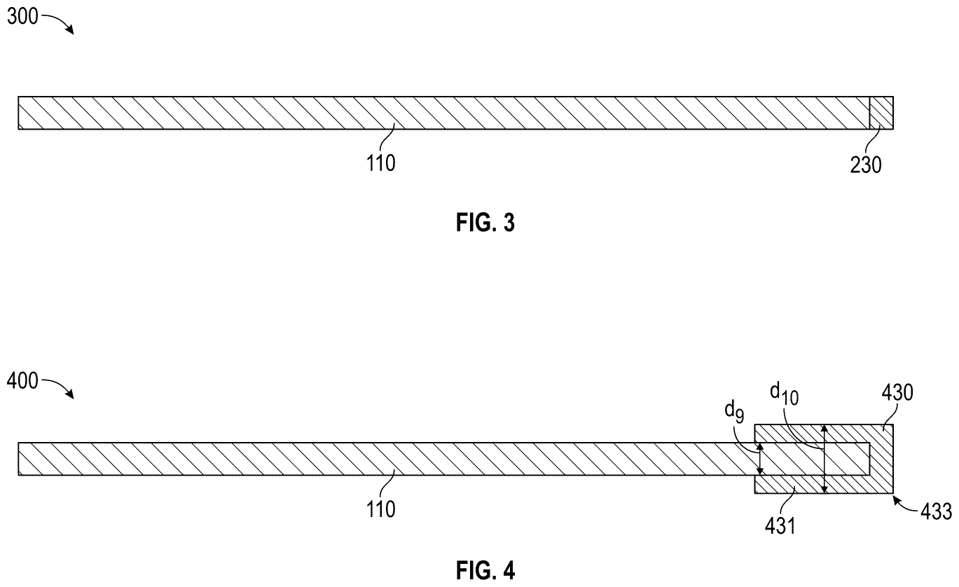

The surgical system 100 includes a connector (e.g., port adaptor 114), an optical fiber system (described in further detail with respect to FIG. 2A, FIG. 2B, FIG. 3, and FIG. 4) including an optical fiber 110, an optical fiber cable 111, and the probe 108. The optical fiber 110 may be at least partially housed inside the optical fiber cable 111. A distal end of the optical fiber cable 111 couples to the probe 108 and a proximal end of the optical fiber cable 111 couples to a port adaptor 114. In some cases, the optical fiber 110 may include more than one fiber. The optical fiber 110 may include a germanate glass (e.g., GeO₂), a fluoride glass (e.g., ZBLAN, aluminum fluoride, etc.), a photonic crystal fiber, a hollow core fiber, or other material. The optical fiber 110 may have a transmissibility in the range of about 1 μm to about 10 μm, such as about the 3 μm wavelength range. In some embodiments, the numerical aperture of the optical fiber 110 is from about 0.02 and about 0.5. The optical fiber 110 may have a diameter d₁ (shown in FIG. 2A and FIG. 2B). The diameter d₁ may be greater than 100 μm, such as from about 100 μm to about 500 μm, such as about 200 μm to about 300 μm.

The port adaptor 114 couples to an optical port of laser system 102. The optical fiber 110 extends through the port adaptor 114 toward the optical port. The port adaptor 114 may include a ferrule 115 with an opening into which a proximal end of optical fiber 110 and a proximal end of the optical fiber cable 111 are inserted. The proximal end of optical fiber 110 includes an interface (e.g., a proximal assembly 240, described below in further detail) upon which laser beams from surgical laser system 102 may be focused when the proximal end of optical fiber 110 is inserted into the ferrule 115. The interface of optical fiber 110 comprises the exposed proximal ends of the one or more cores to which laser beams may be directed.

The probe 108 may be a tissue removal probe, such as a cataract removal probe, a vitrectomy probe, etc., or a probe used when treating retinal tears and detachments. The probe 108 includes a probe body 112 and a probe tip 140. The optical fiber 110 extends through the probe body 112 to a distal end portion 145 of the probe tip 140. Optical fiber 110 delivers the laser beams through probe 108, which guides the laser beams to the vitreous 122, the retina 120, the crystalline lens 121 of a patient's eye 125, or the like. The probe body 112 and the probe tip 140 house and protect the distal end of optical fiber 110. A distal end portion 145 of the probe tip 140 may also contain a protective window (e.g., protective window 230 described in further detail herein) in an opening in a distal end portion 145 of the probe tip 140. The distal end portion 145 may also contain a lens between the protective window and the distal end of the optical fiber 110. In some embodiments, the protective window may itself be a lens that focuses or guides the beams on the retina 120, the vitreous 122, or the crystalline lens 121 and protects the distal end of the optical fiber 110. The protective window 230 may be cylindrical, spherical, of hemispherical. The probe 108 may be a hand-held component for the surgeon to manipulate as necessary during the surgery.

In some examples, laser system 102 may also propagate an illumination beam onto an interface of optical fiber 110 (e.g., which may also include a proximal end of a cladding that holds the cores within optical fiber 110) in order to illuminate the inside of the eye, especially areas of the retina 120 or crystalline lens 121 that are to be treated. In certain aspects, an illumination source generating the illumination beam may be any suitable type of illumination source, including but not limited to an LED light source, a laser light source, or an incoherent light source. An illumination beam from the illumination source is transmitted through the optical fiber 110 and emitted from a distal end of the optical fiber 110. In embodiments where the optical fiber 110 is a single-core fiber, the illumination beam may be transmitted along the single core.

FIG. 2A illustrates an optical fiber system 200A having a connector window 250A within a proximal ferrule 245. FIG. 2B illustrates an optical fiber system 200B having a connector window 250B at a proximal end of a proximal assembly 240B. FIGS. 2A and 2B illustrate the optical fiber system 200A and optical fiber system 200B, respectively, without the accompanying housing components, e.g., the port adaptor 114, the optical fiber cable 111, and the probe 108. The optical fiber systems 200A and 200B include the optical fiber 110, the protective window 230 (e.g., a distal window), a distal ferrule 235, and a proximal assembly (e.g., a connector).

The protective window 230 is disposed at the distal end of the optical fiber 110. The protective window 230 may be housed within the probe 108. For example, the protective window 230 may be housed within the distal end portion 145 of the probe tip 140. The protective window 230 may include a sapphire material, a diamond material, a ruby material, a quartz material, a yttrium aluminum garnet (YAG) material, or a combination thereof.

The protective window 230 may have a thickness t₁. The thickness t₁ may be less than about 10 mm (millimeters), such as less than about 5 mm, such as less than about 1 mm. The thickness t₁ of less than about 10 mm enables more efficient transmission of light. The protective window 230 may have a diameter d₂. The diameter d₂ may be greater than about 100 μm, such as from about 100 μm to about 500 μm, such as about 200 μm to about 300 μm. In some embodiments, the diameter d₂ is approximately equal to the diameter d₁ of optical fiber 110. In other embodiments, the diameter d₂ may be greater than the diameter d₁.

The protective window 230 protects the distal end of the optical fiber 110 from moisture and the thermo-mechanical pressures associated with surgery/treatment. The protective window 230 may have a hardness from about 8 to about 10 on the Mohs scale and a thermal property of about 1 w/m.k (watts per meter-Kelvin) to about 50 w/m.k. The hardness of the protective window 230 reduces or eliminates ablation of the optical fiber 110, allowing for more efficient and accurate projection of the laser beams. Further, the hardness of the protective window 230 reduces damage that may be caused as a result of micro-explosions at the distal end of the optical fiber. The protective window 230 may have bulk transmission properties of more than about 80%. The transmission properties of the protective window 230 accurately guide the laser beams to the desired portions of the patient's eye 125.

The distal ferrule 235 may be housed within the distal end portion 145 of the probe tip 140 and disposed around (e.g., annularly surrounds) a distal region 232 of the optical fiber 110. The distal ferrule 235 may be or include a metallic tube, a ceramic tube, a sapphire tube, or similar material. In embodiments where the distal ferrule 235 is a metallic tube, the metallic tube includes materials such as stainless steel, titanium, a nickel-cobalt alloy, or combinations thereof. In embodiments where the distal ferrule 235 is a ceramic tube, the ceramic tube includes materials such as aluminum oxides.

The distal ferrule 235 may have an inner diameter d₃ and an outer diameter d₄. The inner diameter d₃ may be greater than about 100 μm, such as from about 100 μm to about 500 μm, such as about 200 μm to about 300 μm. The inner diameter d₃ may be greater than the diameter d₁ of the optical fiber 110. The outer diameter d₄ may be from about 200 μm to about 1000 μm.

The protective window 230 may be press-fit or brazed into the distal ferrule 235 to secure the protective window 230 to a distal end of the optical fiber 110. The seal between the protective window 230 and the distal ferrule 235 is a hermetic seal that prevents moisture from diffusing into the distal ferrule 235. Additionally, during the mate/demate cycles, the interface between the protective window 230 and the optical fiber 110 may be exposed to mechanical abuse. The distal ferrule 235 protects the interface from the mechanical abuse of the mate/demate cycles. Although FIGS. 2A and 2B include the distal ferrule 235, the optical fiber system 200A and optical fiber system 200B may not include the distal ferrule 235, e.g., the optical fiber 110 is a bare optical fiber.

As shown in FIG. 2B, the proximal end of the distal ferrule 235 may be sealed with a first seal 260. The first seal 260 may be one of an adhesive seal (e.g., 353N or thermopolymers), a glass solder seal, or a metallized fiber seal for soldering/brazing to the distal ferrule 235. The first seal 260 hermetically seals and protects the optical fiber 110 disposed within the distal ferrule 235. The hermetic seal prevents exposure of the optical fiber 110 to the thermo-mechanical stresses of the surgery and prevents moisture from diffusing into the distal ferrule 235.

As shown in FIG. 2A, the proximal assembly 240A includes a proximal ferrule 245 and a connector window 250A (e.g., a proximal window). As shown in FIG. 2B, the proximal assembly 240B includes a proximal ferrule 245 and a connector window 250B (e.g., a proximal window). The proximal assembly 240A may be housed within the port adaptor 114. The proximal assembly 240A of optical fiber system 200A and the proximal assembly 240B of optical fiber system 200B may be used as the interface between the laser system 102 and the optical fiber system 200A and optical fiber system 200B, respectively. The connector window 250A, 250B may focus the laser beams provided by the laser system 102 on the proximal end of the optical fiber 110. In some embodiments, the optical fiber system 200A and the optical fiber system 200B may have both the connector window 250A and the connector window 250B, respectively, and the protective window 230. In some embodiments, the optical fiber system 200A and the optical fiber system 200B may have the connector window 250A and the connector window 250B, respectively, but not the protective window 230. In some embodiments, the optical fiber system 200A and the optical fiber system 200B may have the protective window 230 but not the connector window 250A or the connector window 250B, respectively.

The proximal ferrule 245 may be disposed around (e.g., annularly surrounds) a proximal region 242 of the optical fiber 110 that couples to the laser system 102. The proximal ferrule 245 may include fused silica, a ceramic tube, or a metallic tube. In embodiments where the proximal ferrule 245 is a metallic tube, the metallic tube includes materials such as stainless steel, titanium, a nickel-cobalt alloy, or combinations thereof. In embodiments where the proximal ferrule 245 is a ceramic tube, the ceramic tube includes materials such as aluminum oxides. The proximal ferrule 245 may have an outer diameter d₅ and an inner diameter d₆. The outer diameter d₅ may be from about 200 μm to about 1000 μm. The inner diameter d₆ may be greater than about 100 μm, such as from about 100 μm to about 500 μm, such as about 200 μm to about 300 μm. The inner diameter d₆ may be greater than or equal to the diameter d₁ of the optical fiber 110.

The connector window 250A and connector window 250B are disposed at the proximal end of the optical fiber 110. The connector window 250A and connector window 250B may include materials such as fused silica, a sapphire material, a diamond material, a ruby material, a quartz material, a YAG material, or a combination thereof. The material of the connector window 250A and connector window 250B may be the same material as the protective window 230. In other embodiments, the material of the connector window 250A and connector window 250B are a different material from the protective window 230, e.g., the percentage of sapphire or diamond may be different.

The connector window 250A and connector window 250B may have a thickness t₂. The thickness t₂ may be less than about 10 mm, such as less than about 5 mm, such as less than about 1 mm. A thickness t₂ of less than about 10 mm enables more efficient transmission of light. As shown in FIG. 2A, the connector window 250A may have a diameter d₇. The diameter d₇ may be greater than about 100 μm, such as from about 100 μm to about 500 μm, such as about 200 μm to about 300 μm. The diameter d₇ is approximately equal to the diameter d₁ of the optical fiber 110. The connector window 250A is disposed at the proximal end of optical fiber 110 within the proximal ferrule 245. In certain embodiments, the connector window 250A may be fused to the optical fiber 110. In certain embodiments, the connector window 250A may be bonded to the proximal ferrule 245 with an adhesive (e.g., a fiber bond). A gap 246A may exist between the connector window 250A and the laser system 102.

As shown in FIG. 2B, the connector window 250B may have a diameter d₈. The diameter d₈ may be greater than about 100 μm, such as from about 100 μm to about 500 μm, such as about 200 μm to about 300 μm. The diameter d₈ may be greater than the diameter d₁ of the optical fiber 110. The connector window 250B is disposed at the proximal end of the optical fiber 110 and the proximal ferrule 245. The connector window 250B may be bonded to the proximal end of the proximal ferrule 245, such as by brazing or adhesive. In FIG. 2B a gap 246B may exist that separates the connector window 250B from the proximal end of the optical fiber 110. In some embodiments, the proximal end of the optical fiber 110 is coplanar with the proximal end of the proximal ferrule 245. The proximal end of the optical fiber 110 may be in contact with the connector window 250B. The connector window 250B may be coupled to the proximal end of the optical fiber 110 by, for example, brazing, direct fusion, or direct deposition.

As shown in FIG. 2B, the distal end of the proximal assembly 240 may include a second seal 270. The second seal 270 may be one of an adhesive seal, a glass solder seal, or a metallized fiber seal for soldering/brazing to the distal ferrule 235. The second seal 270 hermetically seals and protects the optical fiber 110 disposed within the proximal ferrule 245. The hermetic seal prevents exposure of the optical fiber 110 to the thermo-mechanical stresses of the surgery and prevents moisture from diffusing into the proximal ferrule 245.

As further shown in FIG. 2B, a portion of the optical fiber 110 between the proximal assembly 240 and the distal ferrule 235 may be surrounded by a cladding 280. The cladding 280 may include fused silica. The refractive index of the cladding 280 is less than the refractive index of the optical fiber 110. The refractive index of the cladding 280 being lower than the refractive index of the optical fiber 110 allows the light to be guided through the optical fiber 110. The cladding 280 may further protect the optical fiber 110 from exposure to moisture during the surgery/treatment. In some embodiments, the proximal assembly 240 may not include the proximal ferrule 245, e.g., the optical fiber 110 is a bare optical fiber.

FIG. 3 illustrates a distal end of an optical fiber system 300 without a distal ferrule 235. The optical fiber system 300 may be used with a proximal assembly 240 from either optical fiber system 200A or optical fiber system 200B. The optical fiber system 300 includes the optical fiber 110 and a protective window 230. The protective window 230 is disposed at the distal end of the optical fiber 110. The protective window 230 may be formed using direct deposition (e.g., laser pulsed deposition) or direct fusion of the protective window 230 onto the distal end of the optical fiber 110.

During direct deposition, a base material (e.g., sapphire or diamond) is pulsated with a laser to create a fume of the base material. The distal end of the optical fiber 110 is placed in proximity to the fume, causing redeposition of the base material at the distal end of the optical fiber 110 to form the protective window 230. During direct fusion, the distal end of the optical fiber 110 is exposed to energy (e.g., heat), causing the distal end of the optical fiber 110 to melt. The protective window 230 is then pressed against the melted portion of optical fiber 110 to secure the protective window 230 to the distal end of the optical fiber 110.

FIG. 4 illustrates a distal end of an optical fiber system 400 having a distal cap 433. The optical fiber system 400 may be used with a proximal assembly 240 from either optical fiber system 200A or optical fiber system 200B. The optical fiber system 400 includes the optical fiber 110 and the distal cap 433. The distal cap 433 includes an annular sidewall 431 and protective window 430. The annular sidewall 431 surrounds a distal region of the optical fiber 110. The distal cap 433 is secured to the optical fiber 110 using an adhesive seal (e.g., 353N or thermopolymers), a glass solder seal, or a metallized fiber seal for soldering/brazing.

The annular sidewall 431 of the distal cap 433 may be formed by micro-drilling, dry etching, wet etching, and/or other similar approaches. In addition, the distal cap 433 may be a grown sapphire material or grown diamond material. The protective window 430 is disposed at the distal end of the optical fiber 110. The distal end of the optical fiber 110 contacts the protective window 430 of the distal cap 433.

The annular sidewall 431 may include an inner diameter d₉ and an outer diameter d₁₀. The inner diameter d₉ may be greater than about 100 μm, such as from about 100 to about 500 μm, such as about 200 μm to about 300 μm. The inner diameter d₉ may be greater than or equal to the diameter d₁ of the optical fiber. The outer diameter d₁₀ may be from about 200 μm to about 1000 μm.

Note that system 100 and optical fiber systems 200A, 200B, 300 and 400 are only exemplary and may be representative of any suitable surgical laser system. For purposes of clarity, only a select few exemplary components of the system 100 are illustrated and described herein. While the description indicates that the optical fiber 110 and various other components are cylindrical, other shapes are contemplated by this disclosure.

In summation, the embodiments of the present disclosure provide a system in which a protective window (e.g., made of a sapphire material or a diamond material) is disposed at the distal end and/or proximal end of an optical fiber, enabling good transmission characteristics while also protecting the optical fiber from heat and/or moisture. The optical fiber (e.g., made from germanate or fluoride glass) is capable of efficiently guiding a laser beam from a laser source to a desired location on a patient's body, such as the patient's eye.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. An optical fiber system, comprising:
an optical fiber;
a distal window on a distal end of the optical fiber; and
a proximal assembly, the proximal assembly comprising:
a proximal ferrule; and
a proximal window, wherein the distal window and the proximal window comprise a sapphire material or a diamond material.

2. The optical fiber system of claim 1, further comprising a distal ferrule disposed around a distal region of the optical fiber.

3. The optical fiber system of claim 2, wherein the distal window is press-fit or brazed into a distal end of the distal ferrule and contacts the distal end of the optical fiber.

4. The optical fiber system of claim 3, further comprising a first seal at a proximal end of the distal ferrule and a second seal at the distal end of the proximal assembly.

5. The optical fiber system of claim 4, wherein the first seal and the second seal comprise an adhesive seal, a glass solder seal, or a metallized fiber seal.

6. The optical fiber system of claim 1, wherein the optical fiber comprises a germanate glass or fluoride glass.

7. The optical fiber system of claim 1, wherein the proximal ferrule comprises a metallic tube, a ceramic tube, or fused silica.

8. The optical fiber system of claim 1, wherein the proximal window is disposed at a proximal end of the optical fiber within the proximal ferrule.

9. The optical fiber system of claim 1, wherein the proximal window is disposed at a proximal end of the optical fiber and the proximal end of the proximal ferrule.

10. The optical fiber system of claim 1, wherein the distal window is deposited on the distal end of the optical fiber.

11. The optical fiber system of claim 1, wherein the distal window is fused to the distal end of the optical fiber.

12. The optical fiber system of claim 1, further comprising a distal cap, wherein the distal cap comprises the distal window and an annular sidewall, wherein the annular sidewall surrounds a distal region of the optical fiber and the distal end of the optical fiber contacts the distal window of the distal cap.

13. The optical fiber system of claim 1, wherein the distal window has a thickness of less than about 1 mm.

14. The optical fiber system of claim 1, wherein the distal window has a diameter of about 100 μm to about 500 μm.

15. A system, comprising:
a laser system; and
an optical fiber system, the optical fiber system comprising:
an optical fiber;
a distal window on a distal end of the optical fiber; and
a proximal assembly, the proximal assembly comprising:
a proximal ferrule; and
a proximal window, wherein the distal window and the proximal window comprise a sapphire material or a diamond material.
